SEQUENCE LISTING (4 sequences)

SEQ ID NO: 1 - DNA, 58 bp, Artificial Sequence
  single-stranded oligonucleotide
ccgggtgagagttctgggccataaactcgagtttatggcccagaactctcactttttg

SEQ ID NO: 2 - DNA, 58 bp, Artificial Sequence
  single-stranded oligonucleotide
aattcaaaaagtgagagttctgggccataaactcgagtttatggcccagaactctcac

SEQ ID NO: 3 - DNA, 58 bp, Artificial Sequence
  single-stranded oligonucleotide
ccggcgaatccagaaggaacgatttctcgagaaatcgttccttctggattcgtttttg

SEQ ID NO: 4 - DNA, 58 bp, Artificial Sequence
  single-stranded oligonucleotide
aattcaaaaacgaatccagaaggaacgatttctcgagaaatcgttccttctggattcg

(19) United States
(12) Patent Application Publication
(10) Pub. No.: US 2018/0117029 A1
(43) Pub. Date: May 3, 2018

(54) Targeting Metabolic Vulnerability in Triple-Negative Breast Cancer

(71) Applicant: Beth Israel Deaconess Medical Center, Inc., Boston, MA (US)

(72) Inventors: Alex Toker, Boston, MA (US); Kristin K. Brown, Boston, MA (US)

(21) Appl. No.: 15/802,308

(22) Filed: Nov. 2, 2017

Related U.S. Application Data
(60) Provisional application No. 62/417,185, filed on Nov. 3, 2016.

(51) Int. Cl. A61K 31/47; A61K 31/42; A61K 31/277; A61K 31/198; A61K 31/351; A61K 45/06

(52) U.S. Cl. CPC A61K 31/47; A61K 31/42; A61K 31/277; A61K 2300/00; A61K 31/351; A61K 45/06; A61K 31/198

(57) ABSTRACT

Methods for treating cancer in a subject by administering a therapeutically effective amount of a pyrimidine synthesis inhibitor and a genotoxic chemotherapeutic agent. In some embodiments, the cancer is triple negative breast cancer.

### CLAIM OF PRIORITY

This application claims priority under 35 USC § 119(e) to U.S. Provisional Patent Application Ser. No. 62/417,185, filed on Nov. 3, 2016. The entire contents of the foregoing are hereby incorporated by reference.

### TECHNICAL FIELD

Described herein are methods for treating cancer in a subject by administering a therapeutically effective amount of a pyrimidine synthesis inhibitor and a genotoxic chemotherapeutic agent. In some embodiments, the cancer is triple negative breast cancer or ovarian cancer.

### BACKGROUND

Triple-negative breast cancer (TNBC) is a molecularly heterogeneous group of diseases defined by the lack of estrogen receptor (ER), progesterone receptor (PR) and absence of human epidermal growth factor receptor-2 (HER2) amplification. TNBC accounts for about 15% of all breast cancer cases. Consequently, TNBCs are impervious to therapies commonly used in other breast cancer subtypes and treatment options are largely limited to conventional genotoxic chemotherapy agents including doxorubicin (Adriamycin) (1).

### SUMMARY

Chemotherapy resistance is a major barrier to the treatment of triple-negative breast cancer and strategies to circumvent resistance are required. Using in vitro and in vivo metabolic profiling of triple-negative breast cancer cells, we show that an increase in the abundance of pyrimidine nucleotides occurs in response to chemotherapy exposure. Mechanistically, elevation of pyrimidine nucleotides induced by chemotherapy is dependent on increased activity of the de novo pyrimidine synthesis pathway. Pharmacological inhibition of de novo pyrimidine synthesis sensitizes triple-negative breast cancer cells to genotoxic chemotherapy agents by exacerbating DNA damage. Moreover, combined treatment with doxorubicin and leflunomide, a clinically approved inhibitor of the de novo pyrimidine synthesis pathway, induces regression of triple-negative breast cancer xenografts. Thus, the increase in pyrimidine nucleotide levels observed following chemotherapy exposure represents a metabolic vulnerability that can be exploited to enhance the efficacy of chemotherapy for the treatment of triple-negative breast cancer. Collectively, the present studies provide critical evidence to demonstrate that adaptive reprograming of de novo pyrimidine synthesis, induced in response to chemotherapy exposure, can be harnessed and exploited to improve the anti-cancer activity of genotoxic chemotherapy agents for the treatment of cancers such as TNBC.

Thus, provided herein are methods for treating cancer in a subject, comprising administering to the subject a therapeutically effective amount of a pyrimidine synthesis inhibitor and a genotoxic chemotherapeutic agent. In addition, provided are a pyrimidine synthesis inhibitor and a genotoxic chemotherapeutic agent for use in treating cancer, and pharmaceutical compositions comprising a pyrimidine synthesis inhibitor and a genotoxic chemotherapeutic agent, in a physiologically acceptable carrier.

In some embodiments, the pyrimidine synthesis inhibitor is selected from the group consisting of brequinar, leflunomide, teriflunomide, N-(phosphonacetyl)-L-aspartate (PALA), NITD-982, and NITD-102.

In some embodiments, the genotoxic chemotherapeutic agent is selected from the group consisting of alkylating agents, intercalating agents, and DNA replication and repair enzyme inhibitors.

In some embodiments, the intercalating agent is an anthracycline, e.g., selected from the group consisting of daunorubicin, doxorubicin, dactinomycin, idarubicin, nemorubicin, sabarubicin, valrubicin and epirubicin, cisplatin, carboplatin, oxaliplatin, and tetraplatin. In some embodiments, the genotoxic chemotherapeutic agent is doxorubicin.

In some embodiments, the subject has breast, ovarian, endometrial, prostate, bone, colorectal, or non-small cell lung cancer. In some embodiments, the subject has triple negative breast cancer. In some embodiments, the cancer is associated with mutations in PIK3CA and/or PI3K/AKT pathway activation and/or loss of PTEN. In some embodiments, the methods include determining that the subject has cancer associated with mutations in PIK3CA and/or PI3K/AKT pathway activation and/or loss of PTEN, and selecting a subject who has cancer associated with mutations in PIK3CA and/or PI3K/AKT pathway activation and/or loss of PTEN.

In some embodiments, the methods include administering at least one dose of the pyrimidine synthesis inhibitor and at least one dose of the genotoxic chemotherapeutic agent substantially simultaneously, e.g., within 1 day, within 12 hours, within 6 hours, within 2 hours, within 1 hour, within 30 minutes, or within 15 minutes of each other.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-F. Chemotherapy exposure stimulates an increase in pyrimidine nucleotides in TNBC cells. (A) Unbiased hierarchical clustering of relative metabolite abundances in SUM-159PT cells versus SUM-159PT cells treated with 0.5 μM doxorubicin for ten hours. (B) Fold changes in pyrimidine nucleotide abundances, as measured by LC-MS/MS, in vehicle treated SUM-159PT cells versus SUM-159PT cells treated with 0.5 μM doxorubicin for 10 hours. (C) SUM-159PT cells were treated with 0.5 μM doxorubicin or 12.5 μM cisplatin for 10 hours and pyrimidine deoxyribonucleoside triphosphate levels were monitored using a fluorescence-based assay. (D) Schematic of the de novo pyrimidine nucleotide synthesis pathway. (E) Fold changes in dTTP and dCTP levels, following exposure to 0.5 μM doxorubicin for 10 hours in the absence or presence of glutamine (Gln), were monitored using a fluorescence-based assay. (F) Relative isotopic enrichment of L-glutamine (amide-15N) into N-carbamoyl-aspartate was measured by LC-MS/MS in vehicle treated SUM-159PT cells versus SUM-159PT cells treated with 0.5 μM doxorubicin for 4 hours. All error bars represent SEM. N.S. not significant, * P<0.05, ** <0.01, *** P<0.001 by a Student's t-test.

FIGS. 2A-E. Chemotherapy exposure alters the phosphorylation state of CAD to stimulate de novo pyrimidine nucleotide synthesis. (A) SUM-159PT cells were treated with 0.5 μM doxorubicin for the indicated times and the phosphorylation states of CAD, ERK and S6K1 were monitored by immunoblotting. (B) SUM-159PT cells were pre-treated with 5 μM U0126 for 12 hours before a 4 hour exposure to 0.5 μM doxorubicin and the phosphorylation states of CAD, ERK and S6K1 were monitored by immunoblotting. (C) SUM-159PT cells were treated with 0.5 μM doxorubicin for ten hours, in the absence or presence of 5 μM U0126 or 40 μM N-(phosphonacetyl)-1-aspartic acid (PALA), and pyrimidine deoxyribonucleoside triphosphate levels were monitored using a fluorescence-based assay. (D) TNBC cell lines (HCC1143, MDA-MB-468, CAL-51 and MDA-MB-231) were treated with 0.5 μM doxorubicin for 4 hours and changes in CAD phosphorylation were monitored by immunoblotting. (E) TNBC cell lines (HCC1143, MDA-MB-468, CAL51 and MDA-MB-231) were treated with 0.5 μM doxorubicin for 10 hours and pyrimidine deoxyribonucleoside triphosphate levels were monitored using a fluorescence-based assay. All error bars represent SEM. N.S. not significant, * P<0.05, ** P<0.01, *** P<0.001 by a Student's t-test.

FIGS. 3A-G Inhibition of the de novo pyrimidine synthesis pathway sensitizes TNBC cells to genotoxic chemotherapy. (A) SUM-159PT cells were pre-treated with 80 μM N-(phosphonacetyl)-1-aspartic acid (PALA), 0.3 μM brequinar or 20 μM A771726 for 12 hours before exposure to doxorubicin for an additional 48 hours. The percentage of dead cells in the population was determined using a propidium iodide viability assay. (B) The oxygen consumption rate (OCR) of 50,000 SUM-159PT cells treated with vehicle, 0.5 μM doxorubicin (Dox), 20 μM A771726 or the combination of Dox and A771726 for 4 hours was measured using a Seahorse analyzer. (C) SUM-159PT cells were pre-treated with 20 μM A771726 for 12 hours before exposure to Dox for ten hours and pyrimidine deoxyribonucleoside triphosphate levels were monitored using a fluorescence-based assay. (D) SUM-159PT cells were pre-treated with 20 μM A771726 and 100 μM uridine for 12 hours before exposure to doxorubicin for an additional 48 hours. The percentage of dead cells in the population was determined using a propidium iodide viability assay. (E) SUM-159PT cells were pre-treated with 20 μM A771726 for 12 hours before exposure to doxorubicin for 10 hours. Cells were mounted on slides with DAPI after immunostaining with an Alexa-Fluor 647-conjugated p-H2A.X (Ser139) antibody. Images are representative of three independent experiments. (F) SUM-159PT cells were pre-treated with 20 μM A771726 for 12 hours before exposure to cisplatin (12.5 μM), etoposide (40 μM), topotecan (0.625 μM) or paclitaxel (0.5 μM) for an additional 48 hours. The percentage of dead cells in the population was determined using a propidium iodide viability assay. (G) TNBC cell lines (MDA-MB-231, MDA-MB-468, HCC1143, SUM-49PT, CAL-51) were pre-treated with 20 μM A771726 for 16 hours before exposure to doxorubicin for 48 hours. The percentage of dead cells in the population was determined using a propidium iodid viability assay. All error bars represent SEM. N.S. not significant, * P<0.05, ** P<0.01, *** P<0.001 by a Student's t-test.

FIGS. 4A-D. Inhibition of the de novo pyrimidine synthesis pathway in combination with chemotherapy induces regression of TNBC tumor xenografts. (A) Fold changes of pyrimidine nucleotide abundances, as measured by LC-MS/MS, in vehicle treated MDA-MB-231 xenograft tumors versus MDA-MB-231 xenograft tumors treated with 1 mg/kg doxorubicin for 24 hours. (B) MDA-MB-231 xenografts were treated with leflunomide (Lef), doxorubicin (Dox) or a combination of leflunomide and doxorubicin (5 mice per group). Tumors were measured with calipers. (C) Waterfall plot depicting relative tumor volume between the treatment groups 28 days after treatment with vehicle (blue), leflunomide (orange), doxorubicin (purple) or leflunomide and doxorubicin (green). Each bar represents an individual mouse. (D) The body weight of mice treated with vehicle, leflunomide, doxorubicin or leflunomide and doxorubicin was monitored every 7 days for 28 days. No significant changes in body weight were observed during the course of the experiment. All error bars represent SEM. N.S. not significant, ** P<0.01, *** P<0.001 by a Student's t-test.

FIGS. 5A-D. Chemotherapy exposure stimulates an increase in pyrimidine nucleotides in TNBC cells. (A) SUM-159PT cells were treated with 0.5 μM doxorubicin for 10 hours and histone H2A.X phosphorylation was monitored by immunoblotting. (B) SUM-159PT cells were exposed to 0.5 μM doxorubicin for 48 hours and the percentage of dead cells in the population was determined using a propidium iodide viability assay. (C) Fold changes in the abundance of individual metabolites, highlighting pyrimidine nucleotides, as measured by LC-MS/MS in vehicle treated SUM-159PT cells versus SUM-159PT cells treated with 0.5 μM doxorubicin for 10 hours. (D) Fold changes of purine nucleotide abundances as measured by LC-MS/MS in vehicle treated SUM-159PT cells versus SUM-159PT cells treated with 0.5 μM doxorubicin for 10 hours. All error bars represent SEM. * P<0.05, *** P<0.001 by a Student's t-test.

FIGS. 6A-D. Inhibition of the de novo pyrimidine synthesis pathway sensitizes TNBC cells to genotoxic chemotherapy. (A) SUM-159PT cells were infected with empty vector, CAD sh RNA or DHODH shRNA expression vectors. After selection, the expression of CAD and DHODH in the resulting cell lines was monitored by immunoblotting. (B) SUM-159PT cells were infected with empty vector, CAD sh RNA or DHODH sh RNA expression vectors. After selection, the resulting cell lines were treated with 2 μM doxorubicin for 48 hours and the percentage of dead cells in the population was determined using a propidium iodide viability assay. (C) The oxygen consumption rate (OCR) of 50,000 SUM-159PT cells was measured for 30 minutes upon treatment with 0.5 μM doxorubicin or 20 μM A771726 and cells were subsequently challenged with 1 μM oligomycin and 0.5 μM Antimycin A. (D) SUM-159PT cells were pre-treated with 20 μM A771726 for 12 hours before exposure to doxorubicin for 10 hours. Phosphorylation of H2A.X was monitored by immunoblotting. All error bars represent SEM. * P<0.05, ** P<0.01, *** P<0.001 by a Student's t-test.

FIGS. 7A-D: Chemotherapy stimulates an increase in pyrimidine nucleotides in ovarian cancer cells. Shown is syrimidine metabolite abundance in (A) Karumochi, (B) Ovcar3, (C) Ovcar8, and (D) Skov3 ovarian cancer cells treated with cisplatin (CDDP) or doxorubicin (Doxo) for 24 h. n=2.

FIGS. 8A-B: Inhibition of the de novo pyrimidine synthesis pathway sensitizes ovarian cancer cells to genotoxic chemotherapy. (A) OVCAR3 cells pre-treated for 16 h with brequinar prior to 48 h treatment with doxorubicin, n=1. (B) OVCAR8 cells pre-treated for 16 h with brequinar prior to 48 h treatment with doxorubicin, n=3.

### DETAILED DESCRIPTION

Failure to respond to conventional chemotherapy agents is a major barrier to the successful treatment of TNBC. Only approximately 30% of TNBC patients achieve a pathological complete response (pCR) after chemotherapy. For the majority of TNBC patients with residual disease after chemotherapy, high rates of metastatic recurrence are observed and long-term prognosis is poor (2-5). Identification of novel and actionable strategies to sensitize cancer cells to chemotherapy would represent a major advance for the management of TNBC.

Cancer cells exhibit dramatic alterations in cellular metabolism, which support cell growth, proliferation and survival. Indeed, metabolic reprogramming is a recognized hallmark of cancer induced by numerous genetic or epigenetic alterations. Targeting the existing metabolic perturbations that occur in cancer cells has emerged as a promising strategy for cancer therapy (6-8). Recent studies suggest that reprogramming of cellular metabolism is also a component of the highly coordinated response to genotoxic stress (9-11). However, the metabolic response to clinically relevant genotoxic chemotherapy agents is poorly understood.

The present study sought to identify adaptive metabolic reprograming events triggered by chemotherapy exposure that can be targeted to improve the efficacy of chemotherapy for the treatment of TNBC. As described herein, and without wishing to be bound by theory, adaptive metabolic reprogramming of pyrimidine synthesis is an early event that promotes chemotherapy resistance in TNBC cells in vitro and in vivo. As shown herein, genotoxic chemotherapy agents reprogram the de novo pyrimidine biosynthesis pathway to increase the production of nucleotides necessary for DNA repair. Pharmacological inhibition of de novo pyrimidine synthesis sensitizes triple-negative breast cancer cells to clinically relevant chemotherapy agents; inhibition of the de novo pyrimidine synthesis pathway, e.g., with brequinar or leflunomide/A771726, represents a strategy to enhance the in vitro and in vivo sensitivity of TNBC cells to chemotherapy.

### Methods of Treatment

The multifunctional enzyme CAD controls metabolic flux through the de novo pyrimidine synthesis pathway. The catalytic activities of CAD are positively influenced by ERK-dependent and S6K1-dependent phosphorylation events (14-17). In the context of chemotherapy, we find that posttranslational modification of CAD occurs exclusively at the ERK phosphorylation site (Thr456) with no observed changes in phosphorylation at the S6K1 site (Ser1859). It has been proposed that the regulation of CAD by S6K1 represents a mechanism to increase nucleotide production for RNA and DNA synthesis that accompanies cell growth. Here, we propose that the demand for nucleotides to permit DNA repair following genotoxic chemotherapy insult is instead mediated by ERK-dependent regulation of CAD activity. We demonstrate that reprogramming of de novo pyrimidine synthesis is a component of the highly coordinated response to genotoxic stress.

The metabolic pathways that contribute to nucleic acid synthesis have been targeted for cancer therapy for many decades. To this day small molecule inhibitors of these pathways, collectively referred to as antimetabolites, form a central component of therapy regimens in many cancers. As an inhibitor of the de novo pyrimidine synthesis pathway, leflunomide/A771726 is classified as an antimetabolite and is clinically approved for the treatment of a number of autoimmune diseases, in particular rheumatoid arthritis. However, leflunomide has also been shown to possess anti-tumor activity in a number of tumor xenograft models (22, 23). Single-agent leflunomide has been the subject of a number of clinical trials (NCT02509052, NCT01611675, NCT00004071, NCT00003293, NCT00001573, and NCT00003775). As demonstrated herein, when administered in combination with DNA damaging chemotherapy, leflunomide could be repurposed for the treatment of cancers such as TNBC. The present studies support the use of combination therapies with pyrimidine synthesis inhibitors such as leflunomide with genotoxic chemotherapy agents, e.g., in particular doxorubicin, for the treatment of cancers like TNBC.

Pyrimidine Synthesis Inhibitors

Derivatives of the aromatic organic compound pyrimidine include nucleobases found in nucleic acids, namely cytosine, thymine, and uracil. Normal and resting cells recycle or salvage pyrimidine in a manner sufficient to meet their metabolic needs. Cancerous cells, however, proliferate rapidly with a demand for pyrimidines that exceeds the capacity of the salvage pathway, and so must synthesize new pyrimidines via the de novo pathway.

Chemotherapeutic agents that block pyrimidine de novo synthesis reduce nucleobase production during the S phase of the cell cycle, thereby halting normal DNA replication and cell division. Such agents are a subset of anti-metabolite chemotherapeutics, which prevent normal metabolic activity. Inhibiting pyrimidine de novo synthesis can be achieved by metabolic pathway inhibitors. Non-limiting examples of pyrimidine synthesis inhibitors include Brequinar and Leflunomide (trade name ARAVA), as well as active metabolites of Leflunomide, such as Teriflunomide (also known as A771726, trade name AUBAGIO). Leflunomide inhibits the mitochondrial enzyme dihydro-orotate dehydrogenase, which catalyzes the fourth regulated enzymatic step in de novo pyrimidine biosynthesis. Other pyrimidine synthesis inhibitors include N-(phosphonacetyl)-L-aspartate (PALA), a transition-state analog inhibitor of the reaction catalyzed by asparate transcarbamylase; and NITD-982 and its analogue NITD-102, which inhibit dihydroorotate dehydrogenase (DHODH) (Wang et al., Journal of Virology 85(13):6548-56 (2011)). In some embodiments, the pyrimidine synthesis inhibitor specifically inhibits pyrimidine synthesis, i.e., does not inhibit purine synthesis, e.g., is not methotrexate. In some embodiments, the pyrimidine synthesis inhibitor specifically inhibits DHODH.

Genotoxic Chemotherapy Agents

Genotoxic chemotherapy agents are those that work by damaging DNA in cancer cells. DNA integrity is critical for proper cellular function and proliferation. DNA lesions that occur during the S phase of the cell cycle block replication and can lead to DNA double-stranded breaks. Cancerous cells have relaxed DNA repair capabilities and unrepaired DNA gives rise to cell death.

A number of DNA damaging agents are known in the art and can be used in the present methods, including alkylating agents, intercalating agents, and DNA replication and repair enzyme inhibitors. Alkylating agents, which interfere with DNA replication and transcription by modifying DNA bases, include Busulfan, mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin. Intercalating agents function to damage DNA by wedging themselves into the spaces in between nucleotides and include anthracyclines such as Daunorubicin (trade name DAUNOMYCIN), Doxorubicin (trade name ADRIAMYCIN), Dactinomycin, and the platinum-containing agents, e.g., cisplatin (trade name PLATINOL), carboplatin, oxaliplatin, and tetraplatin. Other anthracycline derivatives include Idarubicin, Nemorubicin, Sabarubicin, and Valrubicin (trade name VALSTAR) and Epirubicin (trade name ELLENCE).

Some DNA damaging agents are antimetabolites that masquerade as cytidine, purine or pyrimidine, becoming incorrectly incorporated into DNA. This stops normal development and cell division. Examples include 5-fluorouracil (trade names ADRUCIL, CARAC, EFUDEX and EFUDIX), which acts as a pyrimidine analog and its prodrug doxifluridine; Gemcitabine (trade name GEMZAR), which replaces cytidine; 6-mercaptopurine; capecitabine; 6-thioguanine; cytarabine, and 5-fluorouracil decarbazine.

DNA damaging agents that target enzymes include those that target type I or II topoisomerase. Non-limiting examples of Topoisomerase II inhibitors include mitoxantrone, novobiocin, quinolones (including ciprofloxacin), etoposide and teniposide; inhibitors of Topoisomerase I include irinotecan. Some inhibitors hypomethylate DNA by inhibiting DNA methyltransferase such as decitabine and azacitidine.

Addditional non-limiting examples of chemotherapeutic agents include: bleomycin, vinca alkaloids such as vinblastine, vincristine, vindesine, or vinorelbine. Additional examples of genotoxic anti-cancer treatments are known in the art; see, e.g. the guidelines for therapy from the American Society of Clinical Oncology (ASCO), European Society for Medical Oncology (ESMO), or National Comprehensive Cancer Network (NCCN).

Subjects

Subjects to be treated using the present methods include those who have (e.g., who have been diagnosed with) cancers such as triple-negative breast cancer (TNBC). TNBC is a heterogeneous disease typically diagnosed by a two-step process that includes morphological imaging and biomarker detection, e.g., using immunohistochemistry, in situ hybridization, and/or microarray analysis, and is characterized by the presence of tumors that do not express estrogen receptor (ER) or progesterone receptor (PR) at all, and do not overexpress human epidermal growth factor receptor 2 (HER2). Methods for diagnosing a subject with TNBC are known in the art. See, e.g., Llorca and Viale, Ann Oncol 23(suppl 6):vi19-vi22 (2012); Oakman et al., Breast 19:312-321 (2010); Hammond et al., J Clin Oncol 28:2784-2795 (2010); Wolff et al., J Clin Oncol 25:118-145 (2007); Goldhirsch et al., Ann Oncol 22:1736-1747 (2011); Goldhirsch et al., Ann Oncol 20:1319-1329 (2009). In some embodiments, samples can be considered ER/PR-positive if at least 1% of the tumor cells are immunoreactive, and HER2 positive when uniform intense membrane staining (3+) is present in >30% of invasive tumor cells.

TNBC tumors classified as mesenchymal stem-like (MSL) have one of the lowest response rates to anthracycline-based chemotherapy regimens (24). As shown herein, leflunomide drastically sensitizes xenograft tumors derived from the TNBC MSL cell line MDA-MB-231 to the anthracycline doxorubicin. Moreover, these in vivo studies used a dose of doxorubicin (1 mg/kg) that is equivalent to approximately 0.1 times (one tenth) the recommended human dose based on body surface area. This, coupled with the fact that leflunomide is well tolerated in humans, suggests that combining pyrimidine synthesis inhibitors such as leflunomide with genotoxic chemotherapy agents represents an effective strategy to treat cancers like TNBC, e.g., MSL subtype TNBC.

In some embodiments, the subjects have cancer, e.g., TNBC, associated with mutations in the phosphatidylinositol-4,5-bisphosphate 3-kinase catalytic subunit alpha (PIK3CA) gene (GenBank Acc. Nos. NG 012113.2 RefSeqGene; NM_006218.3 (mRNA) NP 006209.2 (protein)) and/or phosphatidylinositol 3-kinase/Akt (PI3K/AKT) pathway activation and/or loss of PTEN; methods for identifying those subjects are known in the art, see, e.g., Cossu-Rocca et al., PLoS One. 2015 Nov. 5; 10(11):e0141763; Paplomata and O'Regan, Ther Adv Med Oncol. 2014 July; 6(4): 154-166; Massihnia et al., Oncotarget. 2016 Jul. 26. doi: 10.18632/oncotarget. 10858. Other cancers associated with mutations in PIK3CA and PI3K/AKT pathway activation and loss of PTEN can also be treated using the present methods, including ovarian (see, e.g., Eskander and Tewari, Expert Rev Clin Pharmacol. 2014 November; 7(6):847-58; Cheaib et al., Chin J Cancer. 2015 January; 34(1):4-16; and Cai et al., Oncologist. 2014 May; 19(5):528-35); endometrial (see, e.g., Westin et al., Mol Oncol. 2015 October; 9(8):1694-703; Dong et al., J Transl Med. 2014 Aug. 21; 12:231; Markowska et al., Contemp Oncol (Pozn). 2014; 18(3):143-8; and Chen et al., Curr Med Chem. 2014; 21(26):3070-80); prostate (Chen et al., Front Biosci (Landmark Ed). 2016 Jun. 1; 21:1084-91; and Punnoose et al., Br J Cancer. 2015 Oct. 20; 113(8):1225-33); bone malignancies including bone metastases, multiple myeloma, and osteosarcoma (Xi and Chen, J Cell Biochem. 2015 September; 116(9):1837-47); colorectal (Waniczek et al., Pol J Pathol. 2013 April; 64(1):15-20; Mei et al., Ann Oncol. 2016 October; 27(10):1836-48); and non-small cell lung cancer (Perez-Ramirez et al., Pharmacogenomics. 2015 November; 16(16):1843-62).

The present methods can include determining whether a subject has cancer, e.g., TNBC, associated with mutations in PIK3CA and/or PI3K/AKT pathway activation and/or loss of PTEN, e.g., by determining a sequence of PIK3CA, assaying for PI3K/AKT pathway activation (e.g., by detecting levels of phospho-Ser473-AKT) or by determining levels of PTEN (see, e.g., Owonikoko and Khuri, Am Soc Clin Oncol Educ Book. 2013. doi: 10.1200/EdBook_AM.2013.33.e395). These methods include obtaining a sample from a subject, and evaluating the presence of mutations in PIK3CA and/or level of PI3K/AKT pathway activation and/or of PTEN in the sample, and comparing the presence and/or level with one or more references, e.g., a control reference that represents a normal (wild type) sequence of PIK3CA, or a normal level of PI3K/AKT pathway activation and/or of PTEN, e.g., a level in an unaffected subject, or a normal cell from the same subject, and/or a disease reference that represents a sequence or level of associated with cancer, e.g., a level in a subject having cancer, e.g., TNBC, associated with mutations in PIK3CA and/or PI3K/AKT pathway activation and/or loss of PTEN.

As used herein the term “sample”, when referring to the material to be tested for in this embodiment includes inter alia tissue, whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid, but preferably includes tumor cells or tumor tissues.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable sub stance.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of proteins, e.g., PTEN of this invention. (See U.S. Pat. Nos. 5,118,937; 5,045,694; 5,719,060; 6,225,047)

The sequence, presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, Next Generation Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485): 1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to deteremine the sequence of PIK3CA and detect mutations therein.

Measurement of the level of a biomarker can be direct or indirect. For example, the abundance levels of PTEN protein or mRNA can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes are most commonly used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, the presence of mutations in PIK3CA and/or level of PI3K/AKT pathway activation and/or of PTEN is comparable to the presence and/or level in the disease reference, and the subject is selected for the present treatments.

Suitable reference values can be determined using methods known in the art, e.g., using standard clinical trial methodology and statistical analysis. The reference values can have any relevant form. In some cases, the reference comprises a predetermined value for a meaningful level of PI3K/AKT pathway activation and/or of PTEN, e.g., a control reference level that represents a normal level of PI3K/AKT pathway activation and/or of PTEN, e.g., a level in an unaffected subject or a subject who is not at risk of developing a disease described herein, and/or a disease reference that represents a level of the proteins associated with conditions associated with cancer associated with PI3K/AKT pathway activation and/or loss of PTEN.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control normal reference subject does not have a disorder described herein (e.g., does not have cancer associated with PI3K/AKT pathway activation and/or loss of PTEN).

A disease reference subject can be one who has cancer associated with PI3K/AKT pathway activation and/or loss of PTEN.

The predetermined value can depend upon the particular population of subjects (e.g., human subjects) selected. For example, an apparently healthy population may have a different ‘normal’ range of levels of cancer associated with PI3K/AKT pathway activation and/or of PTEN than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art. In characterizing likelihood, or risk, numerous predetermined values can be established.

### Pharmaceutical Compositions and Methods of Administration

The methods described herein can include the use of pharmaceutical compositions comprising a pyrimidine synthesis inhibitor and a genotoxic chemotherapeutic agent as active ingredients. The pyrimidine synthesis inhibitor and genotoxic chemotherapeutic agent can be administered in separate compositions, or in a single combination composition. Such combination compositions are also provided herein.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: A Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

### EXAMPLES

The invention is further described in the following examples, which do not limit the scope of the invention described in the claims.

### Example 1. Adaptive Reprogramming of De Novo Pyrimidine Synthesis is a Metabolic Vulnerability in Triple-Negative Breast Cancer

Methods

The following materials and methods were used in Example 1.

Cell culture: SUM-159PT cells were obtained from Asterand Bioscience and maintained in Ham's F12 medium (Cellgro) containing 5% fetal bovine serum (FBS; Gibco), 1 μg/mL hydrocortisone (Sigma-Aldrich) and 5 μg/mL insulin (Gibco). CAL-51 cells were a gift from K. Polyak (Dana-Farber Cancer Institute, Boston, Mass., USA). All other cell lines were obtained from the American Type Culture Collection (ATCC). MDA-MB-231, MDA-MB-468 and CAL51 cells were maintained in DMEM (Cellgro) containing 10% FBS. Hs578t cells were cultured in DMEM containing 10% FBS and 10 μg/mL insulin. HCC1143 and HCC1806 cells were maintained in RPMI (Cellgro) containing 10% FBS. BT549 cells were cultured in RPMI media containing 10% FBS and 10 μg/mL insulin. Cell lines were authenticated using short tandem repeat (STR) profiling. Cells were maintained in culture for no longer than 4 months and were routinely assayed for mycoplasma contamination.

Chemotherapy agents and inhibitors: Doxorubicin for in vitro experiments was purchased from Cell Signaling Technology; A771726, leflunomide and doxorubicin for in vivo experiments was purchased from Selleck Chemicals; U0126, etoposide, topotecan hydrochloride, cisplatin and paclitaxel were from Tocris; brequinar was purchased from Sigma-Aldrich. N-(phosphonacetyl)-1-aspartic acid (PALA) was obtained from the Drug Synthesis and Chemistry Branch, Developmental Therapeutics Program, Division of Cancer Treatment and Diagnosis, National Cancer Institute; Oligomycin and Antimycin A were from Sigma.

Antibodies: p-H2A.X, H2A.X, p-CAD (S1859), CAD, p-ERK (T202/Y204), ERK1/2, p-S6K (T389) and S6K antibodies were purchased from Cell Signaling Technology. P-CAD (T456) and DHODH antibodies were obtained from Santa-Cruz Biotechnology. An Alexa-Fluor 647-conjugated p-H2A.X (Ser139) antibody was purchased from BD Biosciences.

LC-MS/MS metabolomics profiling: For in vitro studies, SUM-159PT cells were maintained in full growth medium, and fresh medium was added at the time cells were treated with doxorubicin. For metabolite extraction, medium from biological triplicates was aspirated and ice-cold 80% (v/v) methanol was added. Cells and the metabolite-containing supernatants were collected and the insoluble material in lysates was pelleted by centrifugation at 10,000 g for 10 min. The resulting supernatant was evaporated using a refridgerated SpeedVac. For in vivo studies, ice-cold 80% (v/v) methanol was added to flash-frozen tumor tissue. Tissue was homogenized using a TissueLyser (Qiagen). The insoluble material was pelleted by centrifugation at 10,000 g for 10 min. The resulting supernatant was evaporated using a refridgerated SpeedVac. Samples were re-suspended using 20 μl HPLC-grade water for mass spectrometry. Ten microlitres was injected and analysed using a 5500 QTRAP hybrid triple quadrupole mass spectrometer (AB/SCIEX) coupled to a Prominence UFLC HPLC system (Shimadzu) with selected reaction monitoring (SRM). Peak areas from the total ion current for each metabolite SRM transition were integrated using MultiQuant v2.0 software (AB/SCIEX). Data analysis was performed using MetaboAnalyst.

Isotope labeling: Ham's F12 medium lacking glutamine (Sigma-Aldrich) was supplemented with 100 μM L-glutamine (amide-¹⁵N). SUM-159PT cells were treated with doxorubicin for 4 hours, at which point medium containing doxorubicin was aspirated and labeled medium was added to the cells. After 1 hour of labeling, cellular metabolites were extracted as described above and quantified using SRM on a 5500 QTRAP mass spectrometer using a protocol to detect ¹⁵N-labelled isotopologues of metabolites in the de novo pyrimidine synthesis pathway.

Deoxyribonucleoside triphosphate assay: Cells were maintained in full growth medium, and fresh medium was added at the time cells were treated with doxorubicin. In some cases, SUM-159PT cells were switched to Ham's F12 medium lacking glutamine. For metabolite extraction, medium was aspirated and ice-cold 60% (v/v) methanol was added. The insoluble material in lysates was pelleted by centrifugation at 10,000 g for 10 min. The resulting metabolite-containing supernatants were evaporated using a refridgerated SpeedVac. Samples were re-suspended in water and deoxyribonucleside triphosphate levels were measured as previously described (12).

Immunoblotting: Cells were washed with ice-cold PBS and lysed in radioimmunoprecipitation buffer (RIPA; 1% NP-40, 0.5% sodium deoxycholate, 0.1% SDS, 150 mmol/L NaCl, 50 mmol/L Tris-HCl, pH 7.5, protease inhibitor cocktail, 50 nmol/L calyculin A, 1 mmol/L sodium pyrophosphate, and 20 mmol/L sodium fluoride). Cell extracts were cleared by centrifugation and protein concentration was measured with the Bio-Rad DC protein assay. Lysates were then resolved on acrylamide gels by SDS-PAGE and transferred electrophoretically to nitrocellulose membrane (Bio-Rad). Blots were blocked in Tris-buffered saline (TBST) buffer (10 mmol/L Tris-HCl, pH 8, 150 mmol/L NaCl and 0.2% Tween 20) containing 5% (w/v) nonfat dry milk and then incubated with primary antibody overnight. Membranes were incubated with HRP-conjugated secondary antibody and developed using enhanced chemiluminescence substrate (EMD Millipore).

RNA-interference: For shRNA silencing of CAD and DHODH single-stranded oligonucleotides encoding CAD or DHODH target shRNA, and its complement, were synthesized: DHODH sense, 5′-CCG GGT GAG AGT TCT GGG CCA TAA ACT CGA GTT TAT GGC CCA GAA CTC TCA CTT TTT G-3′ (SEQ ID NO:1); DHODH antisense, 5′-AAT TCA AAA AGT GAG AGT TCT GGG CCA TAA ACT CGA GTT TAT GGC CCA GAA CTC TCA C-3′ (SEQ ID NO:2); CAD sense, 5′-CCG GCG AAT CCA GAA GGA ACG ATT TCT CGA GAA ATC GTT CCT TCT GGA TTC GTT TTT G-3′ (SEQ ID NO:3); CAD antisense, 5′-AAT TCA AAA ACG AAT CCA GAA GGA ACG ATT TCT CGA GAA ATC GTT CCT TCT GGA TTC G-3′ (SEQ ID NO:4). The oligonucleotide sense and antisense pair was annealed and inserted into the pLKO.1 backbone. To produce lentiviral supernatants, HEK-293T cells were co-transfected with control or shRNA containing pLKO.1 vectors, VSVG and psPAX2 for 48 hours. Cells expressing shRNA were cultured in medium containing 2 μg/mL puromycin.

Propidium iodide viability assay: Cell viability was assayed with a prodium iodide-based plate reader assay, as previously described (25). Briefly, cells in 96-well plates were treated with a final concentration of 30 μM propidium iodide for 60 minutes at 37° C. The initial fluorescence intensity was measured before digitonin was added to each well at a final concentration of 600 μM. After incubating for 30 minutes at 37° C., the final fluorescence intensity was measured. The fraction of dead cells was calculated by dividing the background-corrected initial fluorescence intensity by the final fluorescence intensity.

Mitochondrial respiration: Mitochondrial respiration was assessed using the Seahorse XFe-96 Analyzer (Seahorse Bioscience). SUM-159PT cells (50,000 cells per well) were treated with vehicle control, 0.5 μM Doxorubicin, 20 μM A771726, or the combination (A771726 & Doxorubicin) for 4 hours in normal media conditions. Following this incubation, media was changed to a non-buffered, serum-free Seahorse Media (Seahorse Bioscience, Catalog #102353) supplemented with 5 mM glucose, 2 mM L-glutamine, and 1 mM sodium pyruvate, and the oxygen consumption rate (OCR) was measured. In addition, OCR was measured for 30 minutes upon drug treatment and cells were subsequently challenged with either 1 μM Oligomycin or 0.5 μM Antimycin A to assess their effect on mitochondrial respiration. All experiments were normalized to cell number.

Immunofluorescence: Cells plated on coverslips were fixed with 2% paraformaldehyde for 10 minutes, permeabilized with 0.5% Triton X-100, and blocked with 1% BSA in 20 mmol/L Tris-HCl, pH 7.5, for 20 minutes. Coverslips were then incubated with Alexa Fluor® 647—conjugated anti-phospho H2A.X 5139 antibody (1:100) for 3 hours. After washing twice with PBS, coverslips were mounted with Prolong Gold antifade reagent containing DAPI (Life Technologies). Images of cells were acquired using a fluorescence microscope (Nikon Eclipse Ti) and digital image analysis software (NIS-Elements, Nikon).

Xenograft studies: Female nude mice (6 weeks old) were purchased from Taconic and maintained and treated under specific pathogen-free conditions. All procedures were approved by the Institutional Animal Care and Use Committee at Beth Israel Deaconess Medical Center (BIDMC) and conform to the federal guidelines for the care and maintenance of laboratory animals. The mice were injected subcutaneously with 4×10⁶ MDA-MB-231 cells in medium containing 50% growth-factor-reduced, phenol red-free Matrigel (Corning). Tumor formation was examined every two to three days for the duration of the experiment. For metabolomics profiling, when tumors reached a size of 5-6 mm in diameter, mice were divided into a control group (n=5 mice) and a treatment group that was exposed to 1 mg/kg doxorubicin (n=5 mice). Tumors were collected and flash-frozen 24 hours after animals were exposed to vehicle or doxorubicin. For combination therapy studies, when tumors reached a size of 5-6 mm in diameter, animals were divided into a control group and treatment groups of leflunomide alone, doxorubicin alone, and leflunomide in combination with doxorubicin (n=5 mice per group). Leflunomide (7.5 mg/kg) was administered on days 1-7, 10, 14, 17, 21 and 24 by i.p. injection. Doxorubicin (1 mg/kg) was administered on days 1, 7, 14 and 21 by i.p. injection. Mice were weighed on days 1, 7, 14, 21 and 28. Tumor volume was calculated using the following equation: Tumor volume=(π/6)(W²)(L), where W represents width, and L represents length.

Results

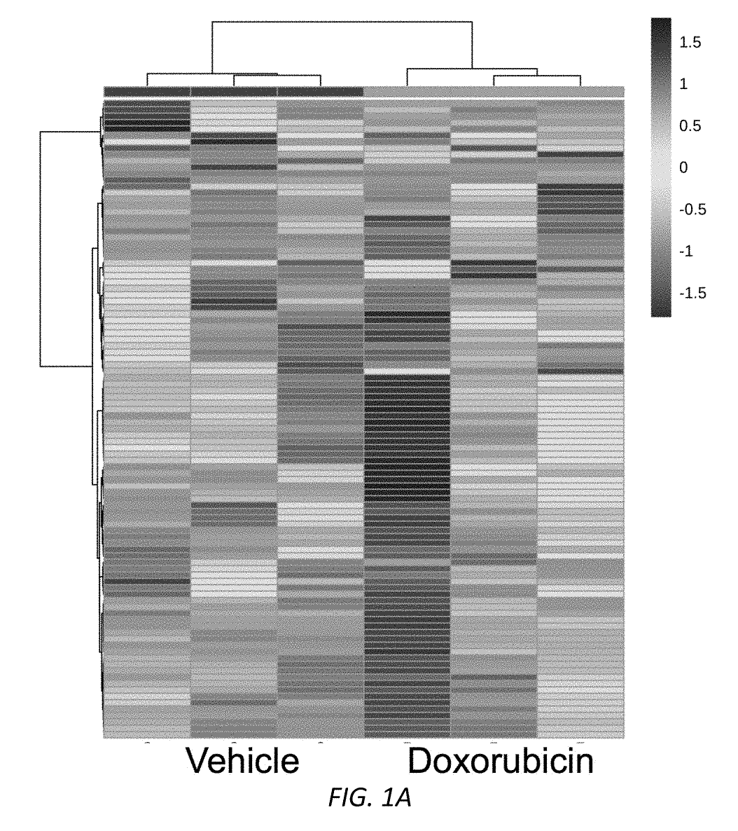

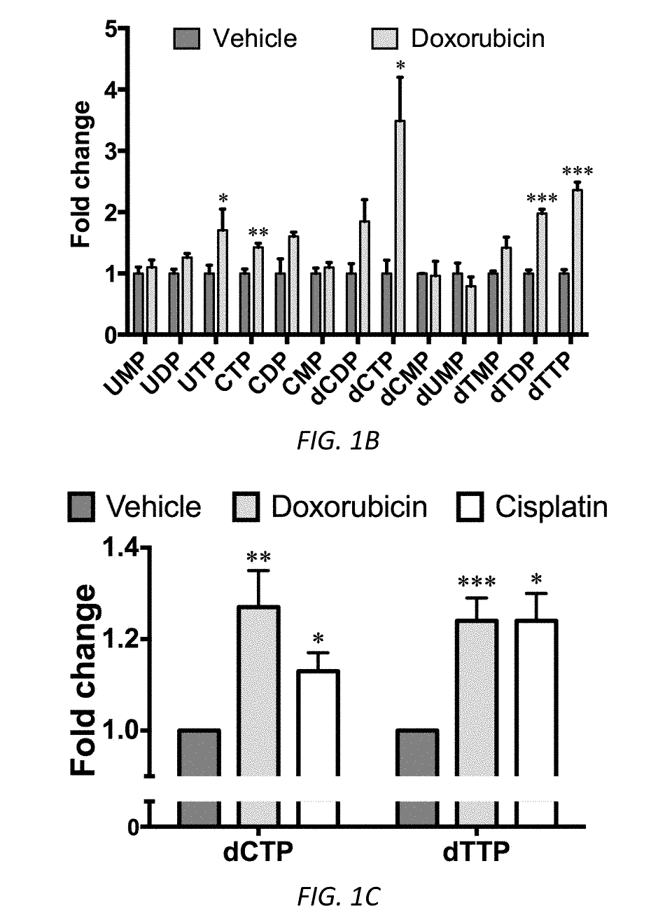

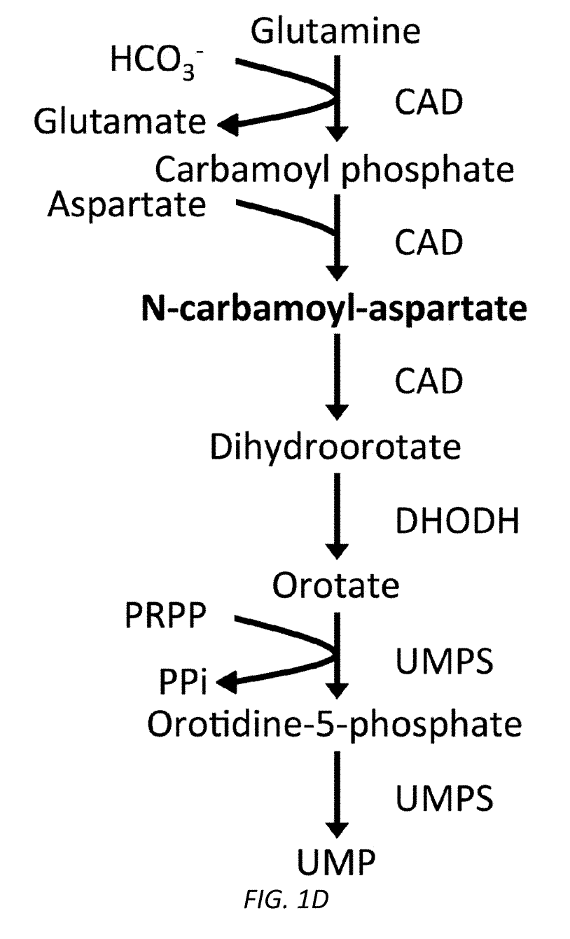

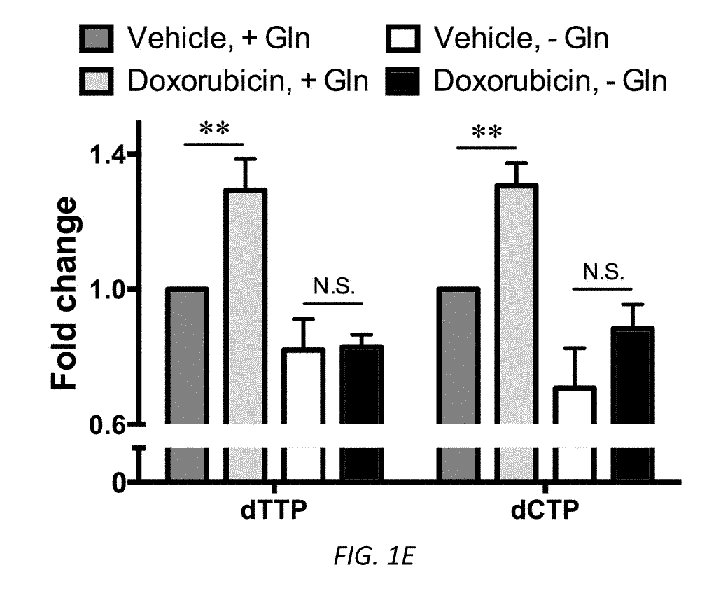

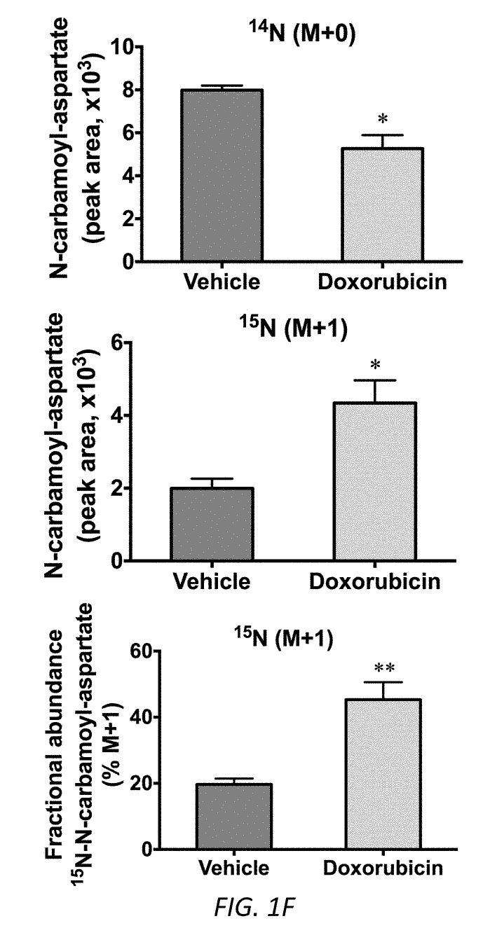

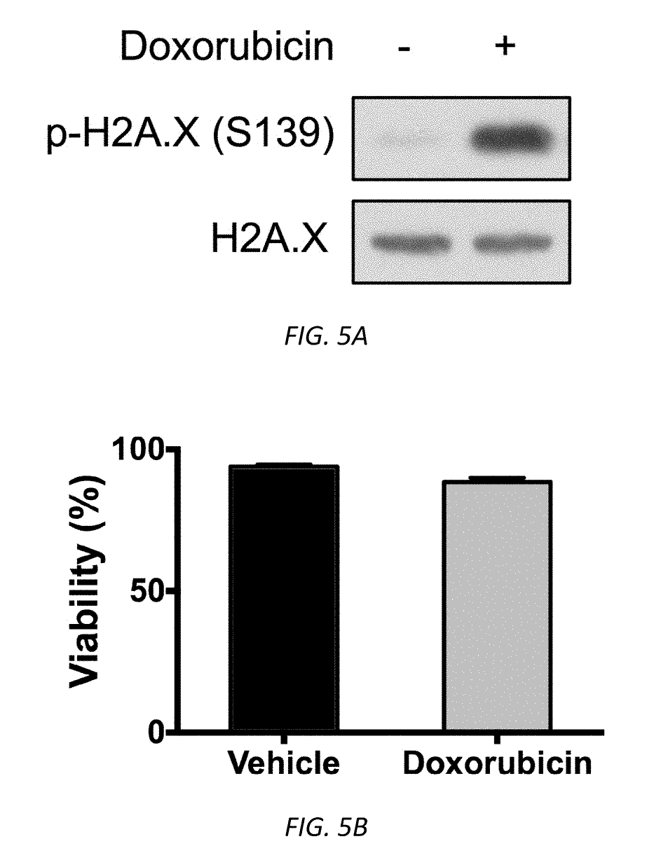

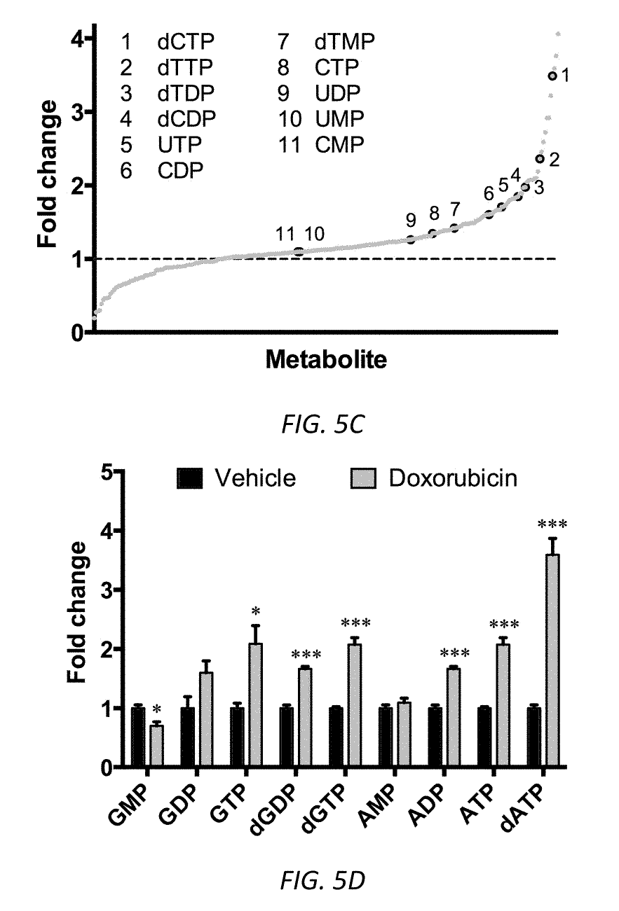

To examine metabolic reprogramming events that influence the cellular response to chemotherapy, we used targeted liquid chromatography-based tandem mass spectrometry (LC-MS/MS) via selected reaction monitoring to examine changes in the steady state metabolomics profile of the TNBC cell line SUM-159PT induced following an acute (10 hour) exposure to doxorubicin. Cells were treated with a concentration of doxorubicin (0.5 μM) that effectively induced DNA damage but had negligible effects on cell viability at later time points (FIGS. 5A and B). Significant differences in the metabolite profiles of untreated cells and cells exposed to doxorubicin were observed (FIG. 1A). Interestingly, doxorubicin increased the abundance of the majority of pyrimidine nucleotide species, including a greater than 2-fold increase in deoxycytidine triphosphate (dCTP) and deoxythymidine triphosphate (dTTP) levels (FIGS. 1B and 5C). Purine nucleotide species were also elevated in treated cells (FIG. 5D). Using a sensitive fluorescence-based assay to specifically monitor intracellular levels of nucleoside triphosphates (12), we confirmed that doxorubicin robustly increased dCTP and dTTP levels (FIG. 1C). Elevated levels of pyrimidine nucleoside triphosphates were also observed when SUM-159PT cells were exposed to cisplatin, a platinum-based genotoxic chemotherapy agent demonstrated to induce anti-tumor responses in a subset of TNBC patients (FIG. 1C) (13). Two pathways contribute to the synthesis of pyrimidine nucleotides; nucleotides can be recycled by a salvage pathway or synthesized via the glutamine-dependent de novo pyrimidine synthesis pathway (FIG. 1D). Depletion of glutamine effectively eliminated the ability of doxorubicin to elevate pyrimidine nucleoside triphosphate levels (FIG. 1E), suggesting that doxorubicin modulates de novo pyrimidine synthesis. To determine whether the effects of doxorubicin on the steady state abundance of pyrimidine nucleotide species was due to changes in metabolic flux through the de novo pyrimidine synthesis pathway, we measured changes in the relative isotopic enrichment of ¹⁵N-glutamine, labeled on the amide nitrogen that is incorporated into the pyrimidine ring. A significant increase in the incorporation of label into N-carbamoyl-aspartate, which is generated in the first committed step of de novo pyrimidine biosynthesis (FIG. 1D), was observed following doxorubicin treatment (FIG. 1F). These data reveal that reprogramming of de novo pyrimidine synthesis, to generate nucleotide precursors required for DNA synthesis and DNA repair, is an adaptive response to chemotherapy.

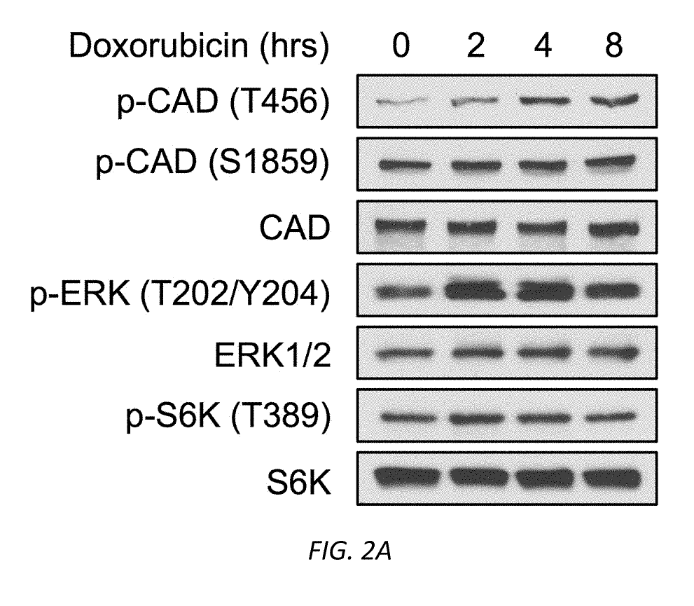

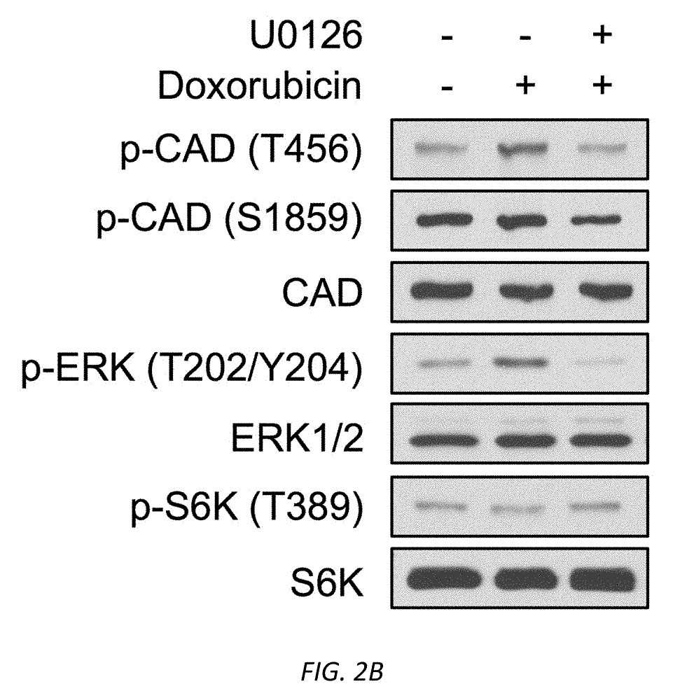

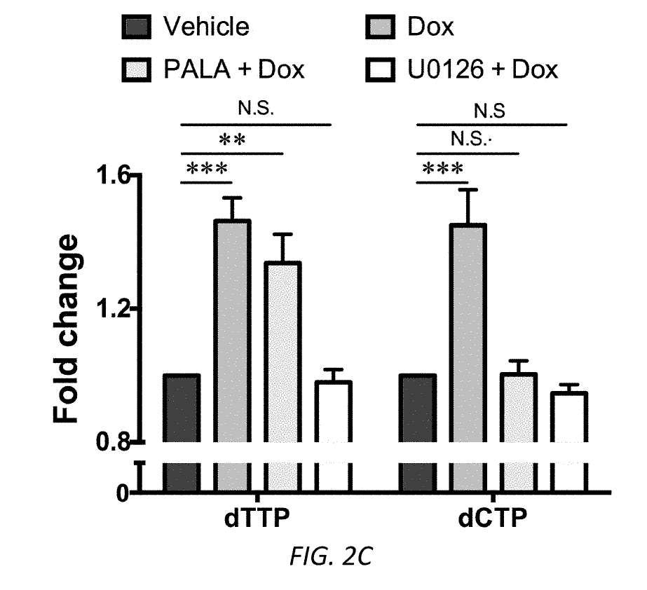

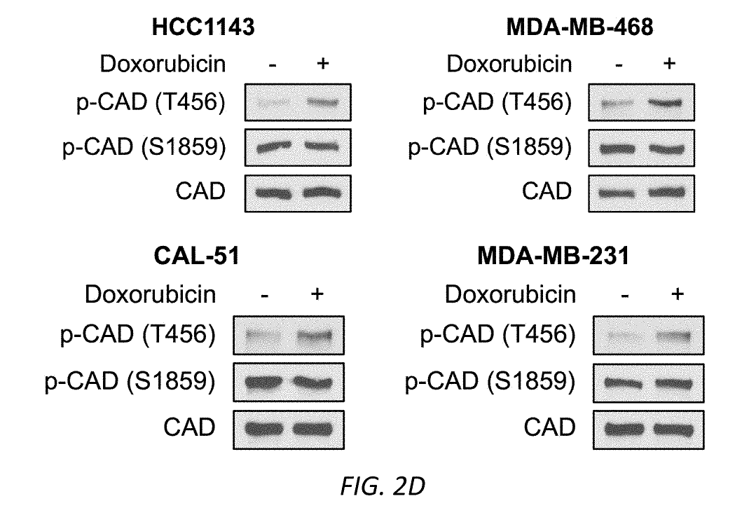

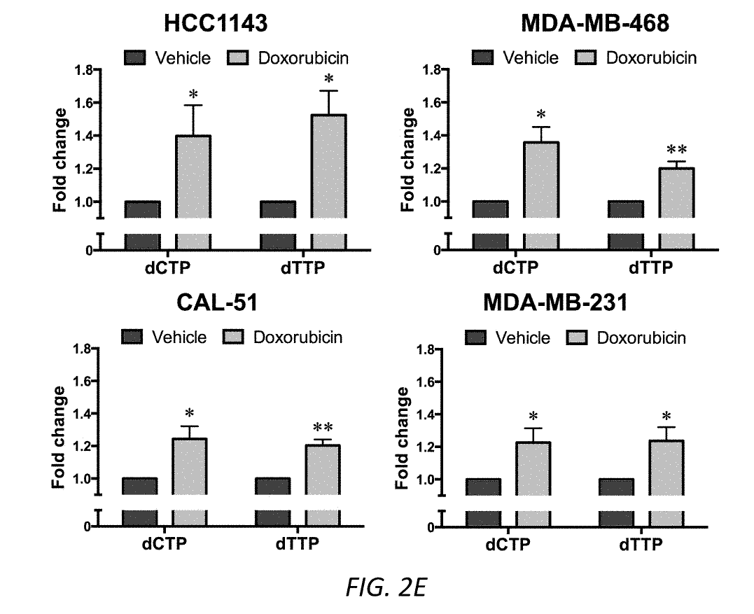

Metabolic flux through the de novo pyrimidine synthesis pathway is controlled by the multifunctional enzyme CAD (carbamoyl-phosphate synthetase 2, aspartate transcarbamoylase, dihydroorotase) (FIG. 1D). The carbamoyl-phosphate synthetase activity of CAD is regulated by protein kinase A (PKA)-dependent and extracellular-signal regulated kinase (ERK)-dependent phosphorylation events (14, 15). Phosphorylation of CAD by ERK relieves feedback inhibition by uridine triphosphate (UTP) and renders CAD more sensitive to activation by phosphoribosyl pyrophosphate (PRPP), thereby promoting pyrimidine synthesis. Additionally, recent studies have revealed that CAD phosphorylation by ribosomal protein S6 kinase 1 (S6K1) increases flux through the de novo pathway in response to growth-promoting signals (16, 17). We observed a striking increase in the phosphorylation of CAD at the ERK site (Thr456) within two hours of treating SUM-159PT cells with low-dose doxorubicin (FIG. 2A). An increase in ERK1/2 phosphorylation was also observed (FIG. 2A). By contrast, there were no measurable changes in the phosphorylation state of S6K1, or the phosphorylation state of CAD at the S6K1 site (Ser1859), in response to doxorubicin exposure (FIG. 2A). The ability of doxorubicin to increase CAD Thr456 phosphorylation was blocked by the highly selective MEK1/2 inhibitor U0126 (FIG. 2B). Interestingly, pre-treatment with U0126 also effectively suppressed the increase in pyrimidine nucleotides observed following doxorubicin exposure (FIG. 2C).

To examine the specific involvement of the de novo pyrimidine synthesis pathway, and more specifically CAD, to the increase in pyrimidine nucleotide levels observed following chemotherapy exposure, SUM-159PT cells were pre-treated with N-(phosphonacetyl)-1-aspartate (PALA) prior to administration of doxorubicin. PALA is a transition state analog of aspartate transcarbamoylase and potent inhibitor of CAD (18). PALA effectively abrogated the increase in dCTP levels induced by doxorubicin treatment (FIG. 2C). PALA did not inhibit doxorubicin-induced alterations in dTTP levels (FIG. 2C), consistent with previous observations that short-term treatment with PALA does not effectively deplete dTTP pools (19). We next examined CAD Thr456 phosphorylation and pyrimidine nucleoside triphosphate levels in additional TNBC cell lines (HCC1143, MDA-MB-468, CAL51 and MDA-MB-231) and universally observed that doxorubicin stimulated CAD Thr456 phosphorylation and increased the abundance of dCTP and dTTP (FIGS. 2D and E). No changes in CAD Ser1859 phosphorylation were observed (FIG. 2D). Together, these data indicate that doxorubicin mediates the posttranslational regulation of CAD activity, and moreover demonstrate that CAD is necessary for increased pyrimidine nucleoside triphosphate production in response to chemotherapy exposure.

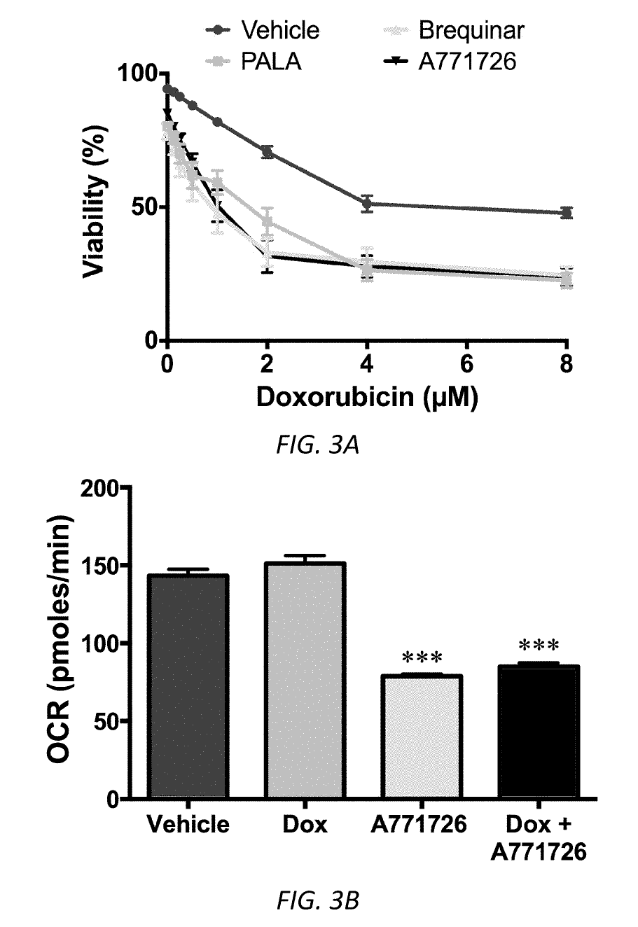

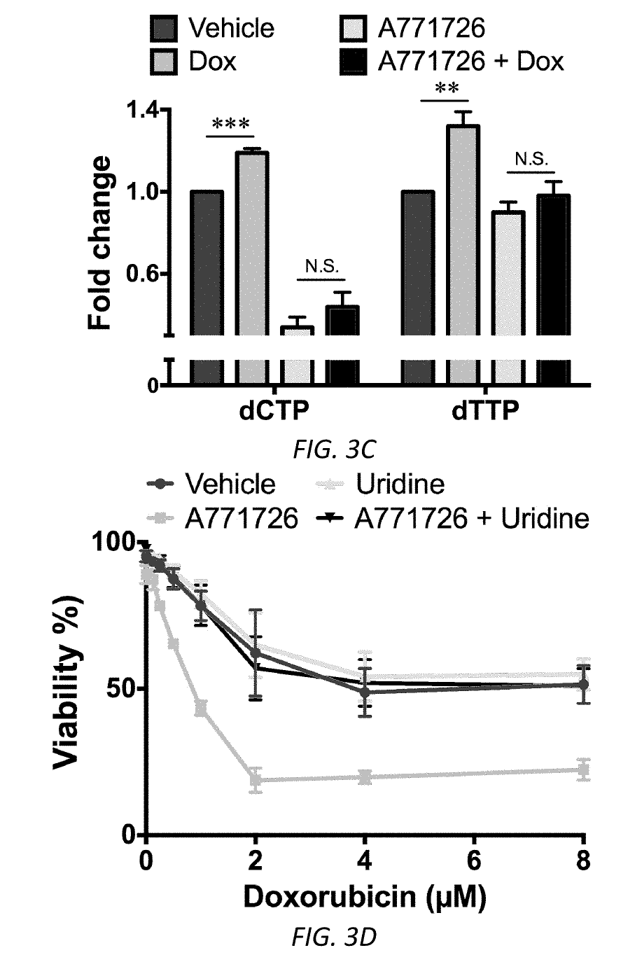

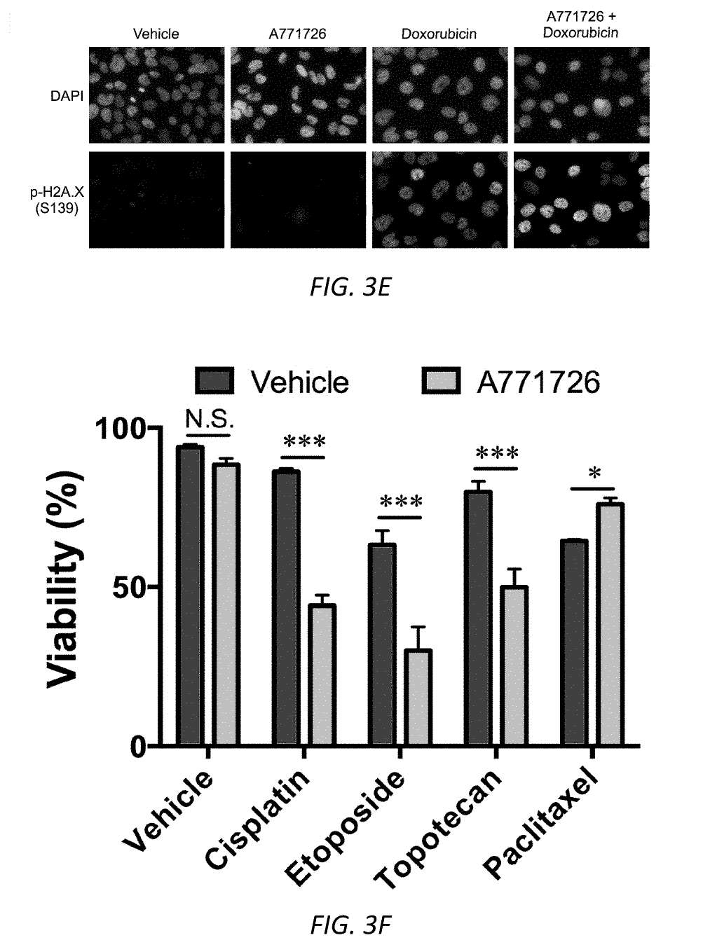

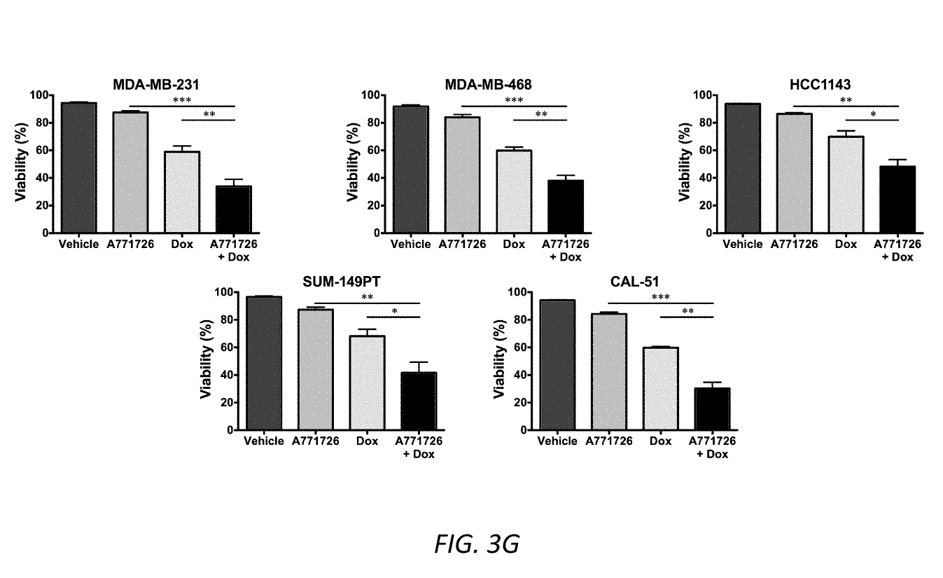

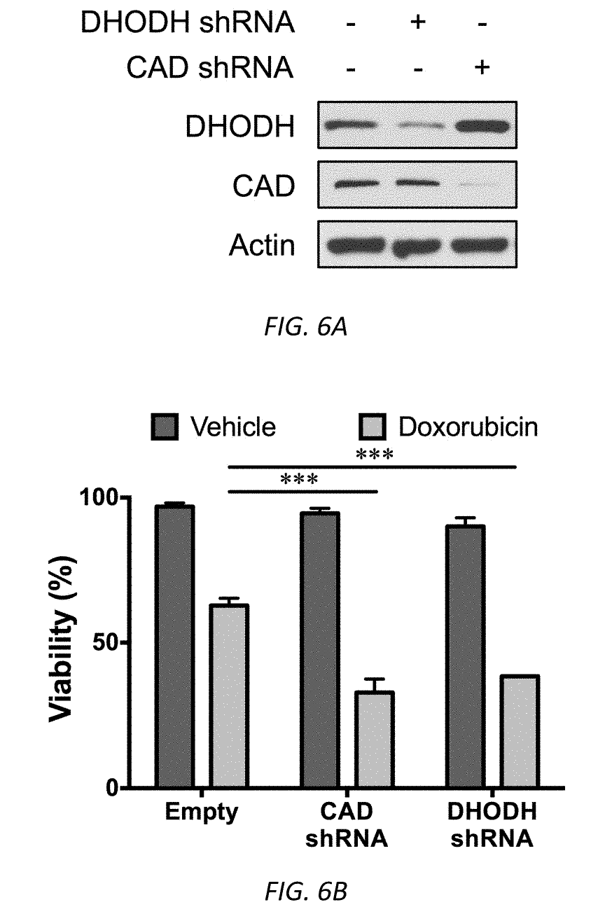

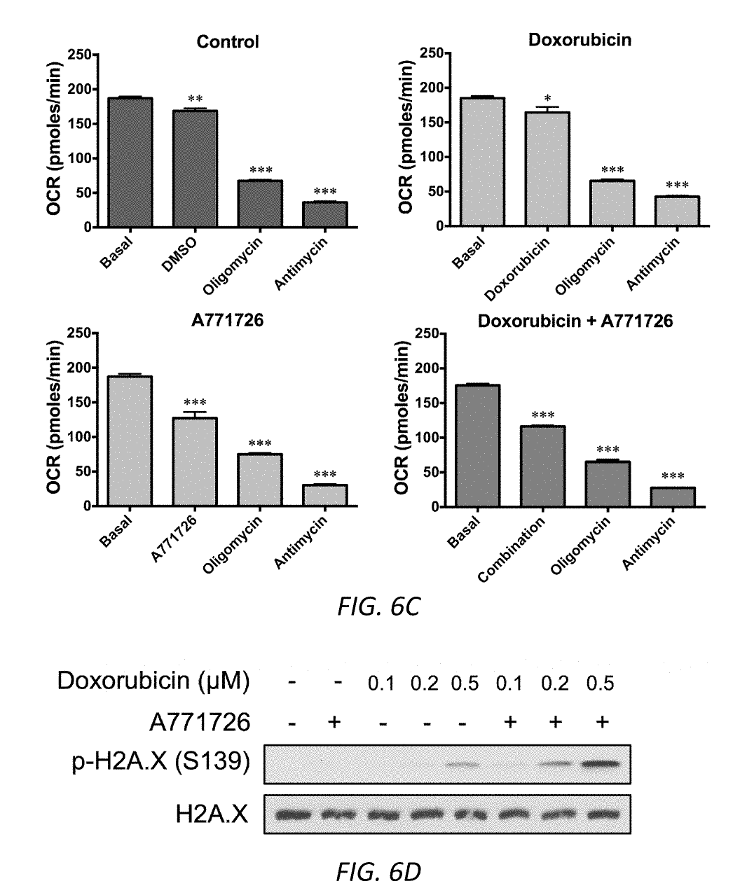

Maintenance of an adequate pool of deoxyribonucleoside triphosphates is essential for DNA replication and DNA repair. It was hypothesized that stimulation of de novo pyrimidine synthesis in response to chemotherapy exposure could therefore represent a metabolic vulnerability that can be exploited to circumvent chemotherapy resistance and thereby enhance the anti-tumor activity of genotoxic chemotherapy agents. Pharmacological inhibition of de novo pyrimidine synthesis has been examined as an anticancer strategy and multiple inhibitors of the pathway have been developed (20). We found that despite exhibiting minimal single-agent activity, PALA and two structurally distinct inhibitors (brequinar and A771726, also known as teriflunomide) of the inner mitochondrial membrane enzyme dihydroorotate dehydrogenase (DHODH), which catalyzes the fourth step of de novo pyrimidine synthesis (FIG. 1D), dramatically sensitized SUM-159PT cells to doxorubicin (FIG. 3A). Moreover, we found that shRNA-mediated depletion of CAD or DHODH sensitized SUM-159PT cells to doxorubicin (FIGS. 6A and B). Given that de novo pyrimidine biosynthesis is coupled to the mitochondrial respiratory chain via DHODH, we examined the oxygen consumption rate (OCR) of SUM-159PT cells 4 hours after doxorubicin exposure. Single agent doxorubicin had a negligible effect on the OCR and did not affect sensitivity to oligomycin and antimycin (FIGS. 3B and 6C), indicating that doxorubicin does not alter mitochondrial respiration. By contrast, 4 hour treatment with A771726 caused a significant decrease in OCR, further validating pharmacological inhibition of DHODH (FIGS. 3B and 6C).

A771726 is the active metabolite of leflunomide, a drug used for the management of autoimmune diseases such as rheumatoid arthritis, which also exhibits some anti-tumor activity (21, 22). Given that leflunomide is widely used in the clinic, and well tolerated in humans, we examined the efficacy of a leflunomide/A771726 and chemotherapy combination. A771726 effectively blocked the increase in dCTP and dTTP levels induced by doxorubicin (FIG. 3C). Next, to confirm that the ability of A771726 to sensitize cells to chemotherapy was due to on-target inhibition of DHODH we performed a rescue experiment with uridine, which contributes to the maintenance of pyrimidine nucleotide pools via the salvage pathway. Application of exogenous uridine completely blocked the efficacy of the combination treatment confirming that A771726 sensitizes cells to chemotherapy by inhibiting de novo pyrimidine synthesis (FIG. 3D). Inhibition of de novo pyrimidine synthesis would be expected to impair the capacity of cells to repair DNA damage. Indeed, pre-treatment with A771726 exacerbated phosphorylation of the histone variant H2A.X, a marker of DNA damage, observed following doxorubicin treatment (FIGS. 3E and 6D). Consistent with the notion that A771726 sensitizes cells to doxorubicin by exacerbating DNA damage and overwhelming the DNA damage response, we observed synergy between A771726 and additional genotoxic chemotherapy agents (cisplatin, etoposide and topotecan) (FIG. 3F). By contrast, A771726 did not sensitize cells to the microtubule destabilizing chemotherapy agent paclitaxel (FIG. 3F). A771726 effectively sensitized additional TNBC cell lines (MDA-MB-231, MDA-MB-468, HCC1143, SUM-149PT, CAL-51) to doxorubicin (FIG. 3G).

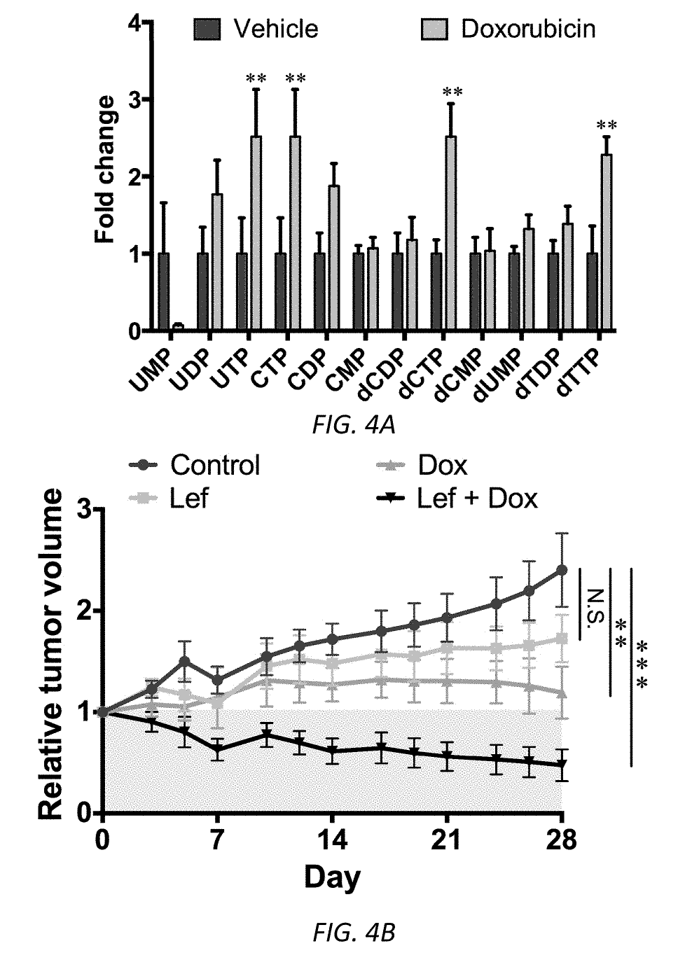

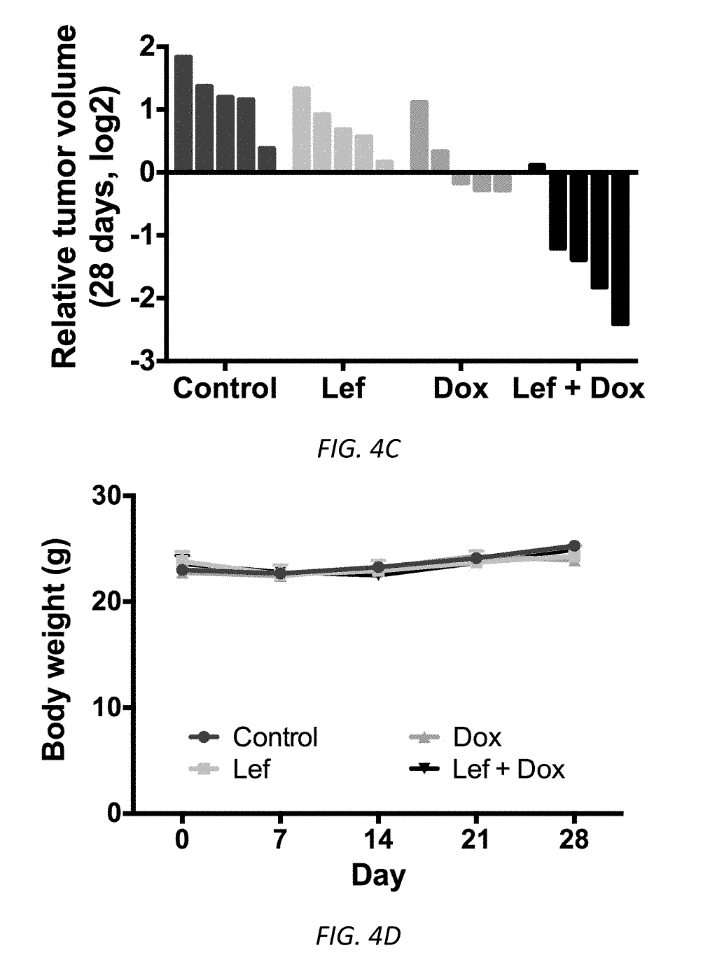

Having demonstrated adaptive reprograming of de novo pyrimidine synthesis in vitro, we sought to examine the conservation of this response in vivo. Mice harboring MDA-MB-231 xenografts were administered doxorubicin for 24 hours. The steady-state metabolomics profile of doxorubicin-treated tumors revealed a significant increase in the abundance of multiple pyrimidine nucleotide species when compared to vehicle-treated tumors, reminiscent of the changes observed in vitro (FIG. 4A). Next, we examined the efficacy of our combination therapy strategy in vivo. Single agent leflunomide did not significantly affect the growth of MDA-MB-231 tumor xenografts in nude mice (FIGS. 4B and 4C). While single agent doxorubicin administration was cytostatic, combined treatment with doxorubicin and leflunomide induced significant tumor regression (FIGS. 4B and 4C). Leflunomide, doxorubicin and the combined therapy did not prevent mice from gaining weight during the course of the experiment (FIG. 4D).

### Example 2. Adaptive Reprogramming of De Novo Pyrimidine Synthesis is a Metabolic Vulnerability in Ovarian Cancer

To expand the applications of our finding, we also tested the effects of chemotherapy on nucleotide metabolism in ovarian cancer. Platinum-based chemotherapy is widely used in treatment of ovarian cancer, thus a novel combination with cisplatin could be readily applied to ovarian cancer treatment.

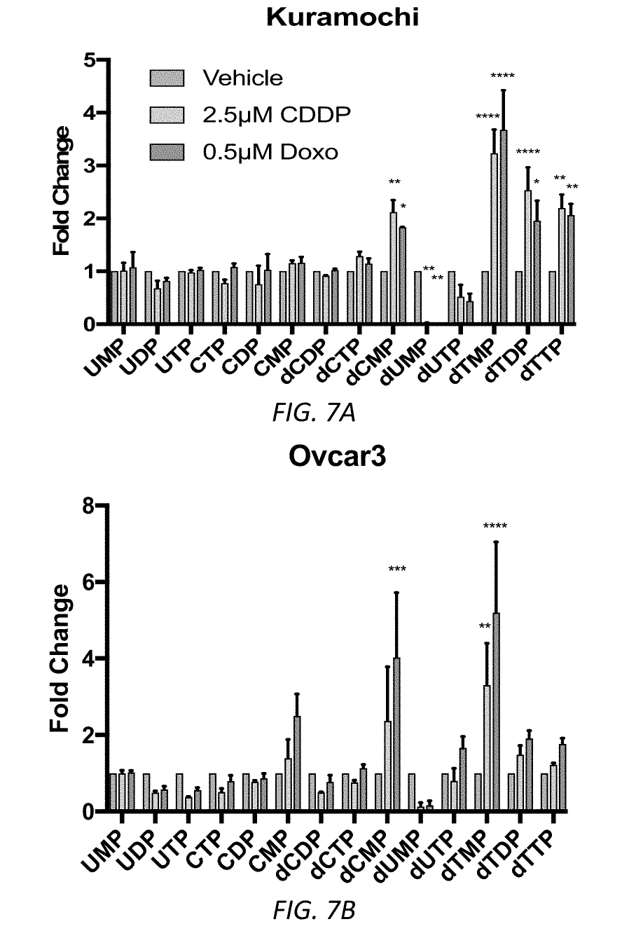

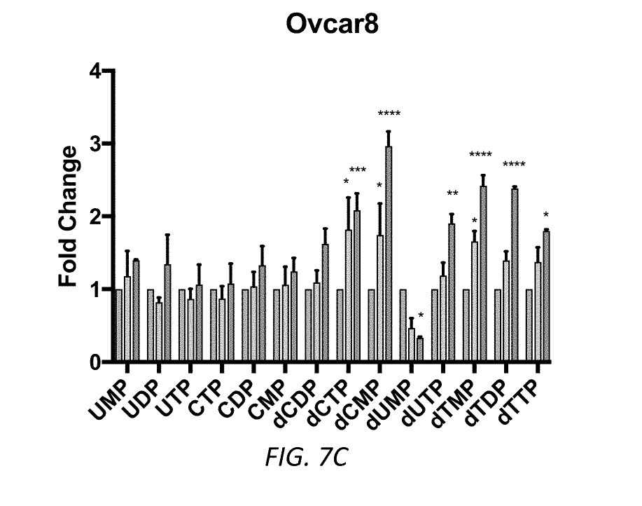

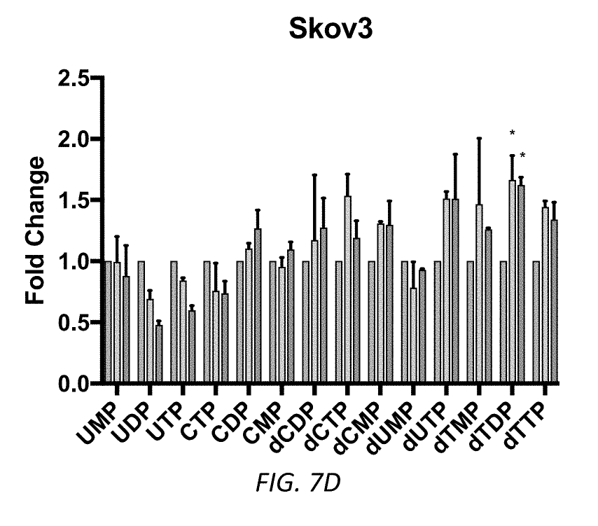

We treated four different human ovarian cancer cell lines with 2.5 μM cisplatin or 0.5 μM doxorubicin for 24 hours and harvested polar metabolites. The samples were profiled using LC-MS/MS for 303 endogenous metabolites. As in breast cancer cells, we observed increases in pyrimidine metabolites (FIGS. 7A-D). The most significantly increased pyrimidines were dCTP, dTMP, dTDP, and dTTP, which increased in multiple lines following cisplatin and doxorubicin treatment.

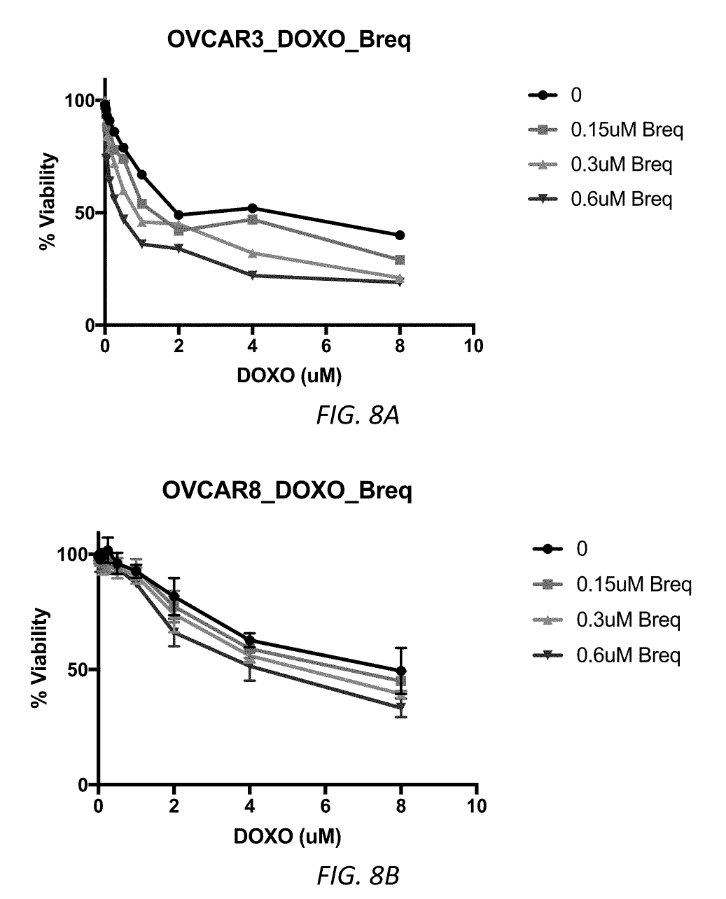

Since ovarian cancers display an increase in pyrimidine nucleotides following chemotherapy, we investigated the ability of pyrimidine synthesis inhibitors to sensitize these cells to chemotherapy. Cells were treated with varying doses of doxorubicin in combination with the DHODH inhibitor brequinar for 48 hours, and viability measured by propidium iodide uptake. As observed in breast cancer cells, brequinar also sensitized ovarian cancer cells to doxorubicin (FIGS. 8A-B).

Together, these data suggest that pyrimidine metabolism is a metabolic vulnerability in triple negative breast cancer and in ovarian cancer. Thus combining inhibitors of pyrimidine metabolism with chemotherapy can increase the tumor-cell killing ability of chemotherapy in multiple cancer cell types.

### REFERENCES

1. Isakoff S J. Triple-negative breast cancer: role of specific chemotherapy agents. Cancer J. 2010; 16:53-61.
2. Guarneri V, Broglio K, Kau S W, Cristofanilli M, Buzdar A U, Valero V, et al. Prognostic value of pathologic complete response after primary chemotherapy in relation to hormone receptor status and other factors. J Clin Oncol. 2006; 24:1037-44.
3. Kuerer H M, Newman L A, Smith T L, Ames F C, Hunt K K, Dhingra K, et al. Clinical course of breast cancer patients with complete pathologic primary tumor and axillary lymph node response to doxorubicin-based neoadjuvant chemotherapy. J Clin Oncol. 1999; 17:460-9.
4. Liedtke C, Mazouni C, Hess K R, Andre F, Tordai A, Mejia J A, et al. Response to neoadjuvant therapy and long-term survival in patients with triple-negative breast cancer. J Clin Oncol. 2008; 26:1275-81.
5. Cortazar P, Zhang L, Untch M, Mehta K, Costantino J P, Wolmark N, et al. Pathological complete response and long-term clinical benefit in breast cancer: the CTNeoBC pooled analysis. Lancet. 2014; 384:164-72.
6. Ward P S, Thompson C B. Metabolic reprogramming: a cancer hallmark even warburg did not anticipate. Cancer Cell. 2012; 21:297-308.
7. Hanahan D, Weinberg R A. Hallmarks of cancer: the next generation. Cell. 2011; 144:646-74.
8. Galluzzi L, Kepp O, Vander Heiden M G, Kroemer G. Metabolic targets for cancer therapy. Nat Rev Drug Discov. 2013; 12:829-46.
9. Cosentino C, Grieco D, Costanzo V. ATM activates the pentose phosphate pathway promoting anti-oxidant defence and DNA repair. Embo J. 2011; 30:546-55.
10. Jeong S M, Xiao C, Finley L W, Lahusen T, Souza A L, Pierce K, et al. SIRT4 has tumor-suppressive activity and regulates the cellular metabolic response to DNA damage by inhibiting mitochondrial glutamine metabolism. Cancer Cell. 2013; 23:450-63.
11. Aird K M, Worth A J, Snyder N W, Lee J V, Sivanand S, Liu Q, et al. ATM couples replication stress and metabolic reprogramming during cellular senescence. Cell Rep. 2015; 11:893-901.
12. Wilson P M, Labonte M J, Russell J, Louie S, Ghobrial A A, Ladner R D. A novel fluorescence-based assay for the rapid detection and quantification of cellular deoxyribonucleoside triphosphates. Nucleic Acids Res. 2011; 39:e112.
13. Silver D P, Richardson A L, Eklund A C, Wang Z C, Szallasi Z, Li Q, et al. Efficacy of neoadjuvant Cisplatin in triple-negative breast cancer. J Clin Oncol. 2010; 28:1145-53.
14. Sigoillot F D, Evans D R, Guy H I. Growth-dependent regulation of mammalian pyrimidine biosynthesis by the protein kinase A and MAPK signaling cascades. J Biol Chem. 2002; 277:15745-51.
15. Graves L M, Guy H I, Kozlowski P, Huang M, Lazarowski E, Pope R M, et al. Regulation of carbamoyl phosphate synthetase by MAP kinase. Nature. 2000; 403:328-32.
16. Ben-Sahra I, Howell J J, Asara J M, Manning B D. Stimulation of de novo pyrimidine synthesis by growth signaling through mTOR and S6K1. Science. 2013; 339:1323-8.
17. Robitaille A M, Christen S, Shimobayashi M, Cornu M, Fava L L, Moes S, et al. Quantitative phosphoproteomics reveal mTORC1 activates de novo pyrimidine synthesis. Science. 2013; 339:1320-3.
18. Collins K D, Stark G R. Aspartate transcarbamylase. Interaction with the transition state analogue N-(phosphonacetyl)-L-aspartate. J Biol Chem. 1971; 246:6599-605.
19. Wadler S, Mao X, Bajaj R, Hallam S, Schwartz E L. N-(phosphonacetyl)-L-aspartate synergistically enhances the cytotoxicity of 5-fluorouracil/interferon-alpha-2a against human colon cancer cell lines. Mol Pharmacol. 1993; 44:1070-6.
20. Christopherson R I, Lyons S D, Wilson P K. Inhibitors of de novo nucleotide biosynthesis as drugs. Acc Chem Res. 2002; 35:961-71.
21. Fragoso Y D, Brooks J B. Leflunomide and teriflunomide: altering the metabolism of pyrimidines for the treatment of autoimmune diseases. Expert Rev Clin Pharmacol. 2015; 8:315-20.
22. White R M, Cech J, Ratanasirintrawoot S, Lin C Y, Rahl P B, Burke C J, et al. DHODH modulates transcriptional elongation in the neural crest and melanoma. Nature. 2011; 471:518-22.
23. Strawn L M, Kabbinavar F, Schwartz D P, Mann E, Shawver L K, Slamon D J, et al. Effects of SU101 in combination with cytotoxic agents on the growth of subcutaneous tumor xenografts. Clin Cancer Res. 2000; 6:2931-40.
24. Masuda H, Baggerly K A, Wang Y, Zhang Y, Gonzalez-Angulo A M, Meric-Bernstam F, et al. Differential response to neoadjuvant chemotherapy among 7 triple-negative breast cancer molecular subtypes. Clin Cancer Res. 2013; 19:5533-40.
25. Zhang L, Mizumoto K, Sato N, Ogawa T, Kusumoto M, Niiyama H, et al. Quantitative determination of apoptotic death in cultured human pancreatic cancer cells by propidium iodide and digitonin. Cancer Lett. 1999; 142:129-37.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating cancer in a subject, the method comprising administering to the subject a therapeutically effective amount of a pyrimidine synthesis inhibitor and a genotoxic chemotherapeutic agent.

2. The method of claim 1, wherein the pyrimidine synthesis inhibitor is selected from the group consisting of brequinar, leflunomide, teriflunomide, N-(phosphonacetyl)-L-aspartate (PALA), NITD-982, and NITD-102.

3. The method of claim 1, wherein the genotoxic chemotherapeutic agent is selected from the group consisting of alkylating agents, intercalating agents, and DNA replication and repair enzyme inhibitors.

4. The method of claim 3, wherein the intercalating agent is an anthracycline.

5. The method of claim 4, wherein the anthracycline is selected from the group consisting of daunorubicin, doxorubicin, dactinomycin, idarubicin, nemorubicin, sabarubicin, valrubicin and epirubicin, cisplatin, carboplatin, oxaliplatin, and tetraplatin.

6. The method of claim 1, wherein the genotoxic chemotherapeutic agent is doxorubicin.

7. The method of claim 1, wherein the subject has breast, ovarian, endometrial, prostate, bone, colorectal, or non-small cell lung cancer.

8. The method of claim 7, wherein the subject has triple negative breast cancer.

9. The method of claim 7, wherein the cancer is associated with mutations in PIK3CA and/or PI3K/AKT pathway activation and/or loss of PTEN.

10. The method of claim 8, wherein the triple negative breast cancer is associated with mutations in PIK3CA and/or PI3K/AKT pathway activation and/or loss of PTEN.

11. The method of claim 1, further comprising determining that the subject has cancer associated with mutations in PIK3CA and/or PI3K/AKT pathway activation and/or loss of PTEN, and selecting a subject who has cancer associated with mutations in PIK3CA and/or PI3K/AKT pathway activation and/or loss of PTEN.

12. The method of claim 1, wherein the method comprises administering at least one dose of the pyrimidine synthesis inhibitor and at least one dose of the genotoxic chemotherapeutic agent substantially simultaneously.

13. The method of claim 12, wherein the method comprises administering at least one dose of the pyrimidine synthesis inhibitor and at least one dose of the genotoxic chemotherapeutic agent within 1 hour of each other.

14. A pharmaceutical composition comprising a pyrimidine synthesis inhibitor and a genotoxic chemotherapeutic agent, in a physiologically acceptable carrier.

15. The pharmaceutical composition of claim 13, wherein the pyrimidine synthesis inhibitor is selected from the group consisting of brequinar, leflunomide, teriflunomide, N-(phosphonacetyl)-L-aspartate (PALA), NITD-982, and NITD-102.

16. The pharmaceutical composition of claim 13, wherein the genotoxic chemotherapeutic agent is selected from the group consisting of alkylating agents, intercalating agents, and DNA replication and repair enzyme inhibitors.

17. The pharmaceutical composition of claim 16, wherein the intercalating agent is an anthracycline.

18. The pharmaceutical composition of claim 17, wherein the anthracycline is selected from the group consisting of daunorubicin, doxorubicin, dactinomycin, idarubicin, nemorubicin, sabarubicin, valrubicin and epirubicin, cisplatin, carboplatin, oxaliplatin, and tetraplatin.

19. The pharmaceutical composition of claim 13, wherein the genotoxic chemotherapeutic agent is doxorubicin.

20. The pharmaceutical composition of claim 13, wherein the genotoxic chemotherapeutic agent is doxorubicin and the pyrimidine synthesis inhibitor is brequinar.
